(19) United States
(12) Patent Application Publication
(10) Pub. No.: US 2020/0345996 A1
(43) Pub. Date: Nov. 5, 2020

(54) LUER ADAPTOR WITH SAFETY LOCK AND RELATED DRAINAGE SYSTEMS

(71) Applicant: Merit Medical Systems, Inc., South Jordan, UT (US)

(72) Inventors: John S. DeMeritt, Saddle River, NJ (US); Christopher Cindrich, Highland, UT (US)

(21) Appl. No.: 16/860,864

(22) Filed: Apr. 28, 2020

Related U.S. Application Data
(60) Provisional application No. 62/841,660, filed on May 1, 2019.

(51) Int. Cl. A61M 39/10; A61M 39/24

(52) U.S. Cl. CPC A61M 39/1011; A61M 2039/1033; A61M 2039/1077; A61M 39/24

(57) ABSTRACT

Medical devices used to transfer fluid are disclosed. More specifically, the present disclosure relates to a locking luer adaptor used to connect medical tubing and various medical catheters, such as a pigtail drainage catheter, to another medical tubing, device, or appliance (such as a drainage system that includes a drainage reservoir or receptacle). The locking luer adaptor may be configured to include a locking spin nut and shroud.

### RELATED APPLICATIONS

This application claims priority to U.S. Provisional Application No. 62/841,660, filed on May 1, 2019 and titled LUER ADAPTOR WITH SAFETY LOCK AND RELATED DRAINAGE SYSTEMS, which is incorporated herein by reference in its entirety.

### TECHNICAL FIELD

The present disclosure relates generally to devices used to transfer fluid (liquid and/or gas), particularly in medical devices. More specifically, the present disclosure relates to a locking luer adaptor that can be used to connect a drainage catheter to a drainage system.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a locking luer adaptor with a safety lock.

FIG. 2 is a perspective, exploded view of the luer adaptor of FIG. 1.

FIG. 3 is a longitudinal, cross-sectional view of the luer adaptor of FIG. 1.

FIG. 4A is a perspective view of a spin nut shroud of the luer adaptor of FIG. 1.

FIG. 4B is a perspective view of a spin nut of the luer adaptor of FIG. 1.

FIG. 5 is a side view of the luer adaptor of FIG. 1, connected to a fluid drainage system and a drainage catheter.

FIG. 6 is a perspective view of another embodiment of a locking luer adaptor.

FIG. 7 is a perspective, exploded view of the luer adaptor of FIG. 6.

FIG. 8 is a longitudinal, cross-sectional view of the luer adaptor of FIG. 6.

FIG. 9A is a perspective view of a spin nut shroud of the luer adaptor of FIG. 6.

FIG. 9B is a perspective view of a spin nut of the luer adaptor of FIG. 6.

FIG. 10 is a side view of the luer adaptor of FIG. 6, connected to a fluid drainage system and a drainage catheter.

FIG. 11A is a perspective view of a spin nut shroud of another embodiment of a locking luer adaptor.

FIG. 11B is a perspective view of a spin nut of the luer adaptor of FIG. 11A.

FIG. 12A is a perspective view of an embodiment of a coupler.

FIG. 12B is another perspective view of the coupler of FIG. 12A.

FIG. 13 is a side view of the coupler of FIG. 12A coupled to a fluid delivery device and a locking luer adaptor.

FIG. 14A is a perspective view of another embodiment of a locking luer adaptor, shown in an engagement state.

FIG. 14B is a perspective view of the luer adaptor of FIG. 14A, shown in a disengagement state.

FIG. 15 is a side view of another locking luer adaptor with an extension tube disposed between a body and a distal portion.

### DETAILED DESCRIPTION

Fluid (e.g., liquid, gas, and/or air) accumulation due to sickness or trauma may develop in areas within a mammalian body not designed to accommodate such accumulation. One particular area prone to abnormal accumulation is between sheets of tissue covering the outside of the lung and lining the chest cavity, known as the pleural space. While a normal functioning pleural space contains approximately 5-20 mL of fluid, fluid turnover occurs on an hourly basis such that approximately 5-10 L of fluid passes through the pleural space every day. Thus, any disruption in fluid turnover may result in an abnormal accumulation or over-accumulation of fluid in the pleural space, known as pleural effusion.

Gas and/or air can also abnormally accumulate in the pleural space due to certain disease processes as well as from trauma, including iatrogenic trauma. The abnormal accumulation of air in the pleural space is called a pneumothorax. The abnormal accumulation of both air and fluid in the pleural space is called a hydropneumothorax. The symptoms of a pleural effusion and/or pneumothorax include dyspnea, tachycardia, cough, breathing difficulty, and chest pain as the lungs are prevented from fully expanding upon breathing.

Pleural effusions can be caused by a wide variety of acute and/or chronic conditions including pneumonia, congestive heart failure, hypoalbuminemia, kidney disease, pulmonary embolism, pancreatitis, cirrhosis, trauma, complications of open-heart surgery, cancer, and malignancy. Drainage of fluid (liquid, gas, and/or air) in the pleural space is desirable to improve cardiopulmonary function, to reduce or eliminate related symptoms, and for diagnostic purposes. This includes acute self-limited conditions such as pneumonia, an exacerbation of a chronic condition such as congestive heart failure, and sometimes-unremitting conditions such as malignant effusions.

There are numerous methods to treat pleural effusion and/or other unwanted fluid accumulation in a mammalian body. Fluid drainage procedures, such as thoracentesis, may be used to provide patient relief. Thoracentesis involves the introduction of a needled catheter into the pleural space through an incision in the skin of the chest wall, and subsequent needle advancement into the chest cavity, after which fluid is drawn out using a syringe or a vacuum source. Drawbacks with this procedure, however, include the fact that the needle may inadvertently puncture the lung, leading to the creation of a pneumothorax from the leakage of air from the injured lung into the pleural space. If the air continues to abnormally accumulate in the pleural space without escaping, it can lead to a tension pneumothorax with cardiovascular collapse, sometimes leading to death. Image guidance (computerized axial tomography or ultrasound) for the performance of thoracentesis has reduced, but not eliminated, this risk. An additional drawback includes the fact that the fluid often re-accumulates in the pleural space after the drainage procedure is performed, such that it may become necessary for a patient to undergo the procedure every few days (or until the underlying cause can be treated).

Percutaneous placed pigtail drainage catheters (which can be placed under image guidance) or surgically placed chest tubes can be used for the short-term drainage of self-limited or medically treatable pleural effusions (congestive heart failure or pneumonia for example). These catheters or tubes are both typically attached to large chest tube drainage systems. Pleurodesis, often performed for chronic malignant effusions, is a procedure in which fluid is prevented from accumulating due to the sealing of the space between pleura with either sterile talc or an antibiotic, after first draining the existing fluid. Another method to treat chronic pleural effusions, such as a malignant effusion, is to surgically implant a tunneled chest tube or catheter such that fluid accumulation can constantly or periodically be removed without repeated procedures. The implanted catheter may be connected to an external catheter or drainage tube by a one-way valve mechanism, which can allow for intermittent fluid drainage via gravity or through the use of a negative pressure source, such as a vacuum.

In the embodiments disclosed herein, transfer of fluid (liquid and/or gas) into and/or out of a mammalian body may be accomplished through fluid transfer devices or luer adaptors. The fluid transfer devices may include a proximal end connectable to a fluid delivery device, such as a syringe, a fluid tubing, etc., or to a fluid drainage device, such as a drainage bag or receptacle.

The fluid transfer devices may also include a distal portion connectable to a catheter inserted into the mammalian body either to deliver fluid or to drain fluid. The distal portion may include a tapered protrusion. A spin nut and a spin shroud may surround the tapered protrusion. The spin shroud may be engageable with the spin nut to rotate the spin nut in a first direction. If desired, the spin shroud may be engageable with the spin nut to rotate the spin nut in both the first direction and a second direction. In other instances, the spin shroud may be engageable with the spin nut to rotate the spin nut in only the first direction. In certain instances, a proximal portion of the luer adaptor is rotatable relative to the distal portion such that the luer adaptor may be connectable to a catheter and substantially unconnectable from the catheter.

Use of a standard universal luer connection can allow for reversible direct connections between various related and unrelated delivery systems including vascular, enteral, respiratory, epidural, and intrathecal medical devices. The locking luer adaptors described herein could be used to better secure an extension tube or other vital medical connection or device such that the connection is either permanent or more difficult to uncouple, preventing an inadvertent or unwanted disconnection of the medical connection or device. In some instances, the locking luer adaptors described herein may be particularly useful to prevent a young child, confused patient, or inexperienced healthcare worker from uncoupling a vital medical connection or machine.

In certain instances, the locking luer adaptor may allow for repurposing of a generic medical device for a specific medical purpose, which cannot be inadvertently changed or modified after a physician or manufacturer makes such a designation. This modular approach is potentially more cost efficient for the production of medical devices by enabling the permanent addition of a more expensive specific component to a readily available base or generic device such as a pigtail catheter or syringe. For example, a specialized valve to control the egress of fluid, a flow rate sensor, or a chemical sensor can be permanently attached to a pigtail drainage catheter, to regulate and or analyze the fluid draining from a body cavity. Similarly, a chemical sensor or filter can be irrevocably connected to a syringe for the aspiration of blood or other bodily fluid, again transforming a generic apparatus into a specific medical device. The locking luer adaptor described herein could also be used to help designate the intended purpose or desired connectivity of an otherwise generic device or catheter.

In other instances, a standard luer system, which is based on a standardized diameter could be irrevocably upsized or downsized for either functional reasons (improved or reduced flow) or for the purpose of designating unique connectivity. The locking luer adaptor could be used to permanently upsize or downsize the diameter of a generic tubing or catheter for a unique purpose, adding specificity based on a predetermined connection diameter which has already been designated for that purpose (enteral feeding for example), i.e. for the next connection in a series of connections, be it tubing, a catheter, or medical device. Similarly, color coded and or embossed segments could be permanently attached to the ends of otherwise generic tubing, drainage catheters, or devices in order to designate a specific purpose, thereby helping to reduce the possibility of unintended connections by healthcare workers. In another instance, the luer adaptors disclosed herein can be used with a ventilator. For instance, the locking luer adaptors described herein may be used to create secure or permanent connections with ventilator components.

It will also be appreciated that the luer adaptors, connectors, and devices disclosed herein can be used in a non-medical application. For example, the luer adaptors, connectors, and devices disclosed herein may be used to transfer fluid (liquid and/or gas) in and/or out of non-living objects, such as mechanical objects or machines. The luer adaptors, connectors, and devices disclosed herein can thus have various uses for transferring various types of fluids (liquids and/or gases).

As detailed below, in some instances the luer adaptor may be used to connect a catheter to a drainage system or a fluid delivery device. Such a system would allow for the intermittent pigtail catheter drainage of pleural fluid without the need for an attached chest tube drainage system, allowing for increased ambulation in the hospital as well as outpatient drainage. The luer adaptor may include a valve configured to prevent fluid from flowing out of the connector and/or gas or air from flowing into the adaptor when the adaptor is in a closed state. The luer adaptor may also include a proprietary configuration to couple with a proprietary connector at a distal end of the drainage system. Exemplary drainage systems that can be used and/or coupled with the valved connector disclosed herein include the Aspira Drainage System, the PleurX Drainage System, and/or one or more components of such drainage systems (e.g., connection interfaces, vacuum bottles, pumps, drainage bags, and/or drainage receptacles, etc.). Other drainage systems and/or components that can be employed and/or coupled with the valved connector disclosed herein include those described in U.S. Pat. Nos. 8,337,475, 8,636,721, and 5,484,401, each of which is incorporated herein by reference in its entirety.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the luer adaptor, the proximal end of the adaptor refers to the end nearest the fluid delivery device or drainage device, and the distal end refers to the opposite end, the end nearest the catheter, such as the pigtail catheter. Thus, if at one or more points in a procedure a physician changes the orientation of a luer adaptor, as used herein, the term “proximal end” always refers to the fluid delivery or drainage device end of the adaptor (even if the distal end is temporarily closer to the physician).

“Fluid” is used in its broadest sense, to refer to any fluid, including both liquids and gases as well as solutions, compounds, suspensions, etc., which generally behave as fluids.

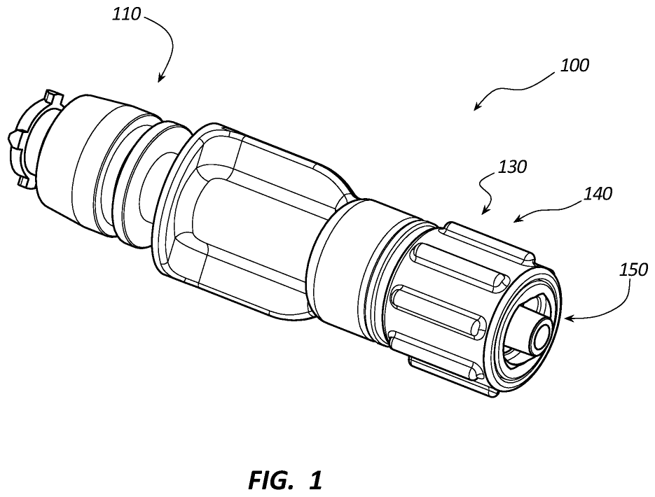

FIGS. 1-15 illustrate views of different embodiments of locking luer adaptors and related components. In certain views, each device may be coupled to, or shown with, additional components not included in other views. Further, in some views only selected components are illustrated and described to provide detail into the relationship of the components. Additionally, some components may be shown in multiple views, but not discussed in connection with every view. It will thus be understood that the disclosure provided in connection with any figure can be relevant and applicable to the disclosure provided in connection with any other figure or embodiment.

FIGS. 1-5 depict one embodiment of a locking luer adaptor 100. As illustrated in FIG. 1, the luer adaptor 100 includes a body 110, a distal member 130, a spin nut shroud 140, and a spin nut 150. The luer adaptor 100 can also be referred to as a connector or an adaptor, and need not include a luer interface.

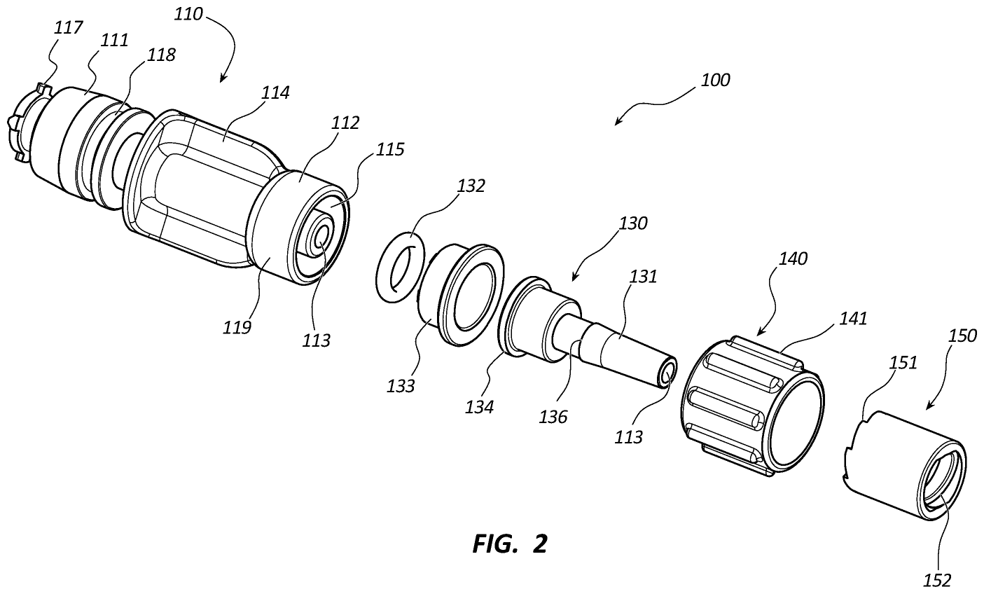

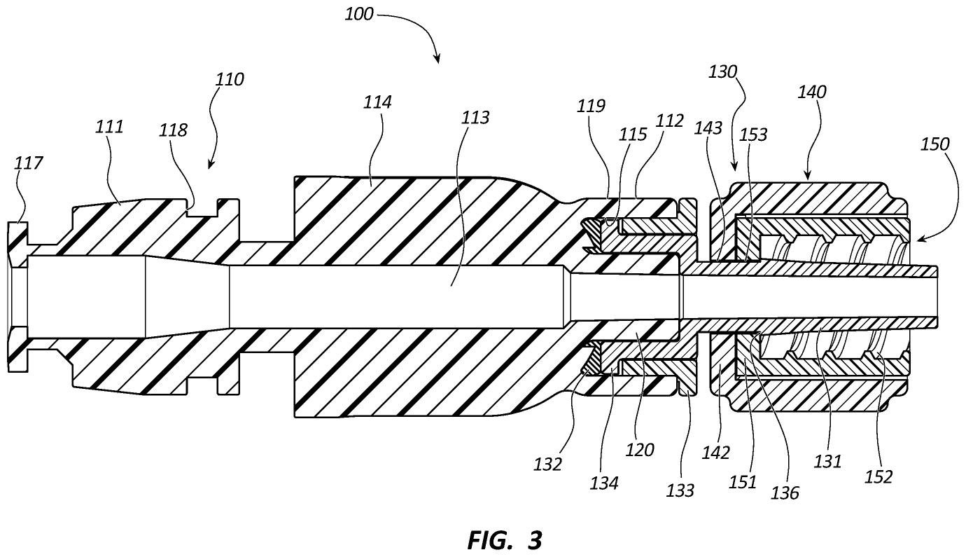

FIG. 2 depicts an exploded view of the luer adaptor 100, and FIG. 3 depicts a cross-sectional view of the luer adaptor 100. As depicted, the body 110 includes a proximal end 111 in fluid communication with a lumen 113. The lumen 113 may extend through the body 110 and the distal member 130 of the luer adaptor 100. In some embodiments, the lumen 113 adjacent the proximal end 111 may be configured as a female taper having a conical 4%-8% taper (e.g., a 6% luer conical taper). This female taper can be configured to sealingly couple with a male taper of a medical delivery or drainage device. In certain embodiments, a valve member may be disposed within the lumen 113 (e.g., such as adjacent to the proximal end 111).

The proximal end 111 may be configured to engage with a medical device. In the illustrated embodiment, the proximal end 111 includes at least one laterally extending anti-rotation protrusion 117 configured to restrict rotation of a connected medical device relative to the luer adaptor 100. In another embodiment, the proximal end 111 may include threads or ears configured to threadingly couple with a male luer lock fitting that includes an internally threaded collar. In further embodiments, the lumen 113 adjacent the proximal end 111 may be configured to couple with medical devices that are specifically configured for a particular medical therapy. For example, the lumen 113 may be configured to receive a male protrusion that is configured to open a valve disposed within the lumen. In another example, the lumen 113 may include a diameter that is configured to receive a male protrusion of a medical device that is configured for deliverance of a specific medicament or fluid. This configuration may reduce the incidence of accidental delivery of a wrong medicament or fluid to a patient.

The body 110 may further include a circumferential engagement groove 118. The engagement groove 118 may be configured to engage with a clip of a medical device to prevent inadvertent proximal displacement of the medical device from the luer adaptor 100. In the illustrated embodiment, the body 110 includes at least one laterally extending wing 114 which can be configured to facilitate rotation of the body 110 and/or luer adaptor 100.

As shown in FIGS. 2-3, the body 110 may include a collar 119 disposed adjacent a distal end 112 of the body 110. The collar 119 may surround a nozzle 120 through which the lumen 113 passes. An annular space 115 may be disposed between the collar 119 and the nozzle 120. The annular space 115 may be configured to receive a portion of the distal member 130.

The distal member 130 of the illustrated embodiment may include one or more of a tapered protrusion 131, a locking collar 133, a seal member 132, a spin nut shroud 140, and a spin nut 150. The tapered protrusion 131 may be configured as a male fitting having a taper of 4%-8% (e.g., 6% luer conical taper) and may be configured to sealingly couple with a female fitting. The tapered protrusion 131 may include a shoulder 136 disposed proximally of a distal end. The shoulder 136 may be configured to restrict the spin nut shroud 140 and the spin nut 150 from distal displacement over the tapered protrusion 131. The tapered protrusion 131 may be partially disposed within the locking collar 133. A flange 134 disposed proximally of the shoulder 136 may be configured to prevent the tapered protrusion 131 from being displaced from the locking collar 133 and to permit relative rotation of the distal member 130 and the body 110. When assembled, the locking collar 133 and a portion of the tapered protrusion 131 may be disposed within the annular space 115. A seal member 132 (e.g., O-ring) may be disposed around the nozzle 120 and between the flange 134 and a proximal end of the collar 119. The seal member 132 may fluidly seal a joint formed between the body 110 and the distal member 130. In some embodiments, the seal member 132 may fluidly seal the joint between the body 110 and the distal member 130 when the body 110 is rotated relative to the distal member 130.

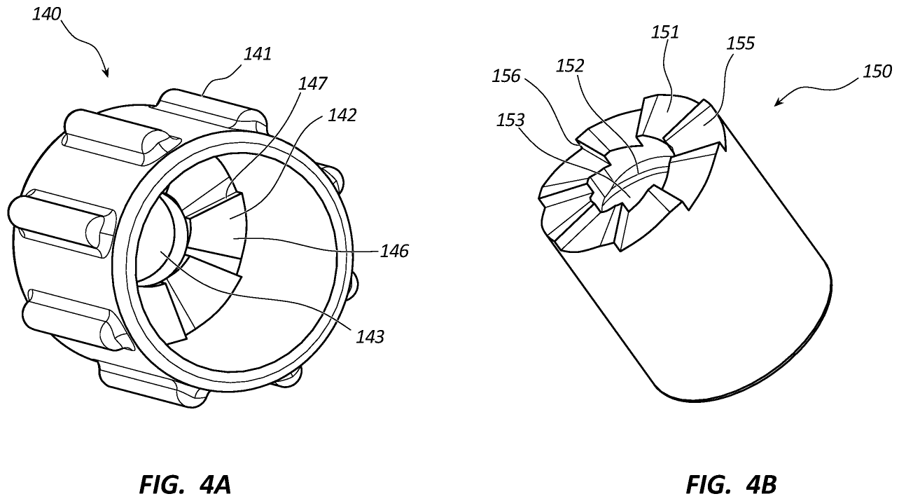

As illustrated in FIG. 4A, the spin nut shroud 140 is generally cylindrical in shape and can be disposed over the spin nut 150. The spin nut shroud 140 may comprise one or more of gripping members 141, a shroud ramp 142, and a shroud passage 143. The gripping members 141 may be configured to enhance gripability of the spin nut shroud 140 when gripped by fingers of a user to rotate the spin nut shroud 140. The gripping members 141 may be of any suitable form. For example, as shown in the illustrated embodiment, the gripping members 141 include a plurality of longitudinally oriented ridges. In other embodiments, the gripping members 141 may include a plurality of bumps or recesses, a textured surface, a compliant surface, etc.

In some embodiments, a shroud ramp 142 may be disposed at a proximal end within the spin nut shroud 140 and circumferentially disposed around the shroud passage 143. The shroud ramp 142 may comprise a shroud ramp surface 146 and a shroud ramp shoulder 147. The shroud ramp surface 146 may be a discontinuously curved helical surface (or a plurality of distinct helical surfaces extending around the spin nut shroud 140). The shroud ramp surface 146 and the shroud ramp shoulder 147 may be configured to engage with the spin nut surface (155 of FIG. 4B) and the spin nut shoulder (156 of FIG. 4B), respectively, such that the spin nut 150 can be rotated in only one direction by the shroud 140. Without limitation, the shroud ramp surface 146 may be angled distally at an angle from about 1 degree to about 45 degrees, from about 10 degrees to about 30 degrees, or from about 15 degrees to about 25 degrees. Other angles are also contemplated. Without limitation, the height of the shroud ramp shoulder 147 may range from about 0.01 inch to about 0.25 inch, from about 0.05 inch to about 0.20 inch, or from about 0.12 inch to about 0.15 inch. Other heights are also contemplated. The spin nut shroud 140 may comprise one, two, three, four, or more shroud ramps 142.

The shroud passage 143 may be disposed at the proximal end of the spin nut shroud 140. A diameter of the shroud passage 143 may be smaller than a diameter of the shoulder 136, such that the spin nut shroud 140 is restricted from distal displacement over the distal member 130. The diameter of the shroud passage 143 may be larger than a portion of the distal member 130 that is proximal to the shoulder 136, such that the spin nut shroud 140 is rotatable relative to the distal member 130.

As illustrated in FIG. 4B, the spin nut 150 is generally cylindrical in shape and can be disposed over the tapered protrusion 131. The spin nut 150 may comprise one or more of a generally smooth outer surface, a spin nut ramp 151, an internal thread 152, and a spin nut passage 153. The internal thread 152 may be configured as a double-start or single start, a right-handed or left handed thread, with a pitch ranging from about 1.0 mm to about 5 mm. For example, the internal thread 152 may be a double-start, right handed threaded having between a 2-3 mm pitch (e.g., or about a 2.5 mm pitch). In other embodiments, the internal thread 152 may be a single-start thread. The internal thread 152 may be configured to threadingly couple with external threads or protrusions of a complementary fitting (e.g., such as a female luer lock fitting).

The spin nut ramp 151 may be disposed at a proximal end of the spin nut 150 and circumferentially disposed around the spin nut passage 153. The spin nut ramp 151 may comprise a spin nut ramp surface 155 and a spin nut ramp shoulder 156. The spin nut ramp surface 155 may be a discontinuously curved helical surface (or a plurality of distinct helical surfaces extending around the spin nut 150). The spin nut ramp surface 155 and the spin nut ramp shoulder 156 may be configured to engage with the shroud ramp surface (146 of FIG. 4A) and the shroud ramp shoulder (147 of FIG. 4A), respectively. Without limitation, the spin nut ramp surface 155 may be angled proximally at an angle from about 1 degree to about 45 degrees, from about 10 degrees to about 30 degrees, or from about 15 degrees to about 25 degrees. Other angles are also contemplated. Without limitation, the height of the spin nut ramp shoulder 156 may range from about 0.01 inch to about 0.25 inch, from about 0.05 inch to about 0.20 inch, or from about 0.12 inch to about 0.15 inch. Other heights are also contemplated. The spin nut shroud 140 may comprise one, two, three, four, or more shroud ramps 142.

The spin nut ramp 151 may be configured to engage with the shroud ramp 142 when the spin nut shroud 140 is rotated in a first direction to rotate the spin nut 150 in a first direction. Additionally, the spin nut ramp 151 may be configured to disengage from the shroud ramp 142 when the spin nut shroud 140 is rotated in a second direction to restrict the spin nut 150 from being rotated in the second direction. In other words, the spin nut ramp surface 155 and the spin nut ramp shoulder 156 are configured to engage with the shroud ramp surface 146 and the shroud ramp shoulder 147 when the spin nut shroud 140 is rotated in a first direction. Also, the spin nut ramp surface 155 and the spin nut shoulder 156 may be configured to disengage from the shroud ramp surface 146 and the shroud ramp shoulder 147 when the spin nut shroud 140 is rotated in the second direction. In some embodiments, a clicking sound will be emitted when the spin nut shroud 140 is rotated in the second direction as the shroud ramp shoulder 147 passes over the spin nut shoulder 156. In some instances, the shroud ramp shoulder 147 passing over the spin nut shoulder 156 may also be felt as it is rotate in the second direction (e.g., a tactile indicator).

The spin nut passage 153 may be disposed at the proximal end of the spin nut 150. A diameter of the spin nut passage 153 may be smaller than a diameter of the shoulder 136, such that the spin nut 150 is restricted from distal displacement over the distal member 130. The diameter of the spin nut passage 153 may also be larger than a portion of the distal member 130 that is proximal to the shoulder 136, such that the spin nut 150 is rotatable relative to the distal member 130.

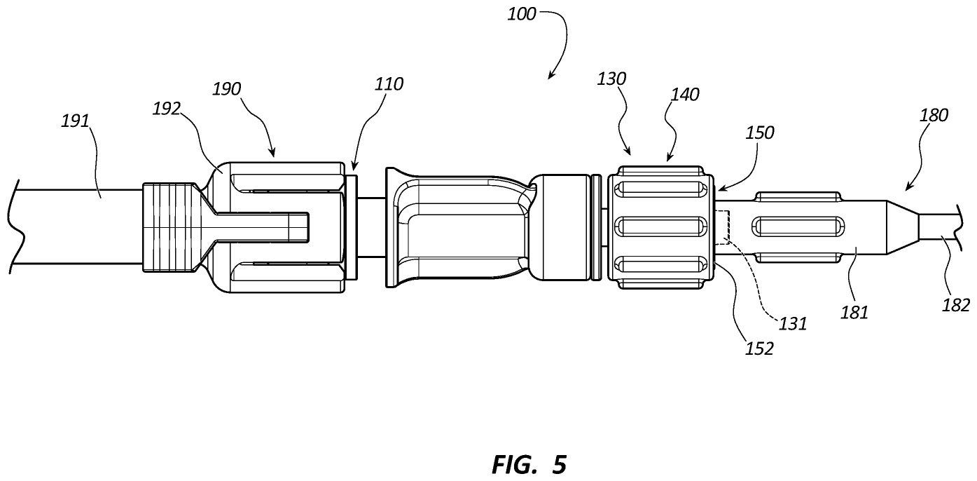

In use, the luer adaptor 100 may be utilized as a component of a fluid delivery or drainage system. In other embodiments, the luer adaptor 100 may be coupled to a pig tail drainage catheter, biliary drainage catheter, a nephrostomy tube, a central venous catheter, a peripheral intravenous catheter, a peripherally inserted central venous catheter, an epidural catheter, an intrathecal catheter, or another type of catheter or device. For example, as depicted in FIG. 5, a fluid drainage system 190 may be coupled to the body 110 such that the luer adaptor 100 is in fluid communication with the fluid drainage system 190. In other embodiments, a fluid delivery system may be coupled to the body 110. The fluid drainage system 190 may comprise a coupler 192, a drainage tube 191, and a drainage receptacle (not shown). Additionally, a drainage catheter 180, such as a pigtail catheter, may be coupled to the distal member 130. The drainage catheter 180 may comprise a catheter body 182 and a catheter hub 181 coupled to the catheter body 182. A distal portion of the catheter body 182 may be inserted into a fluid-filled cavity of a patient. The catheter hub 181 may be configured as a female luer lock fitting. The catheter hub 181 may be coupled to the distal member 130 such that the fluid drainage system 190 is in fluid communication with the fluid-filled cavity through the drainage catheter 180 and the luer adaptor 100.

The catheter hub 181 may be coupled to the distal member 130 when the tapered protrusion 131 is disposed within the catheter hub 181. The spin nut shroud 140 may be displaced distally to engage the shroud ramp 142 with the spin nut ramp 151. When the ramps 142, 151 are engaged, the spin nut shroud 140 may be rotated in the first direction by the user to rotate the spin nut 150 in the first direction. When the spin nut 150 is rotated in the first direction, the thread 152 can threadingly engage with the catheter hub 181, causing the catheter hub 181 to be drawn into the spin nut 150 and over the tapered protrusion 131 to fluidly seal the tapered protrusion 131 and the catheter hub 181.

In some embodiments, the luer adaptor 100 may be configured to be non-releasably coupled to the catheter hub 181, which can prevent inadvertent uncoupling of the luer adaptor 100 from the catheter hub 181. For example, attempted rotation of the spin nut shroud 140 to rotate the spin nut 150 in the second direction to unthread and uncouple the tapered protrusion 131 from the catheter hub 181 may not readily rotate the spin nut 150 as the spin nut ramp 151 disengages from the shroud ramp 142 when the spin nut shroud 140 is rotated in the second direction. In other words, the spin nut shroud 140 disengages from the spin nut 150 when the spin nut shroud 140 is rotated in a second direction allowing the spin nut shroud 140 to rotate freely about the spin nut 150. Additionally, the body 110 may freely rotate relative to the distal member 130 without applying an uncoupling torque force to the distal member 130.

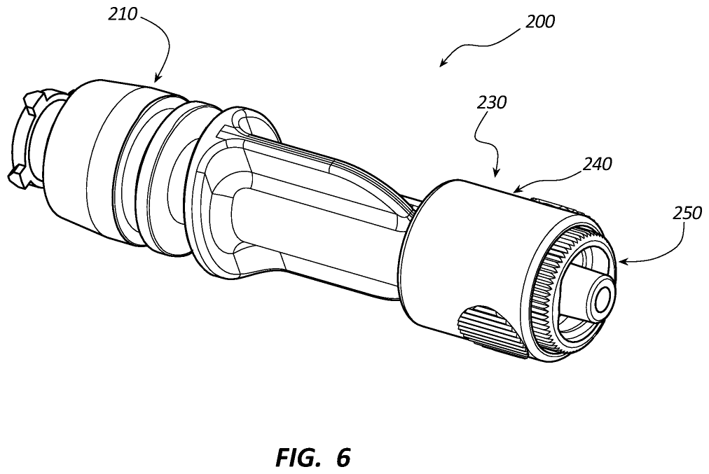

FIGS. 6-10 depict an embodiment of a locking luer adaptor 200 that resembles the luer adaptor 100 described above in certain respects. Accordingly, like features are designated with like reference numerals, with the leading digit incremented to “2.” For example, the embodiment depicted in FIGS. 6-10 includes a distal member 230 that may, in some respects, resemble the distal member 130 of FIG. 1. Relevant disclosure set forth above regarding similarly identified features thus may not be repeated hereafter. Moreover, specific features of the luer adaptor 100 and related components shown in FIGS. 1-5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the luer adaptor 200 and related components depicted in FIGS. 6-10. Any suitable combination of the features, and variations of the same, described with respect to the luer adaptor 100 and related components illustrated in FIGS. 1-5 can be employed with the luer adaptor 200 and related components of FIGS. 6-10, and vice versa. This pattern of disclosure applies equally to further embodiments depicted in subsequent figures and described hereafter, wherein the leading digits may be further incremented.

FIGS. 6-10 depict an embodiment of a locking luer adaptor 200. As illustrated in FIG. 6, the luer adaptor 200 includes a body 210, a distal member 230, a spin nut shroud 240, and a spin nut 250. The luer adaptor 200 can be similar to the luer adaptor 100, except the luer adaptor 200 does not include a collar 119 and associated components for allowing rotation of the body 210. As such, in some embodiments, the body 210 of the luer adaptor 200 may not be configured to rotate relative to the distal member 230. Notwithstanding, if desired, it will be appreciated that such components could be included with the embodiment of FIGS. 6-10.

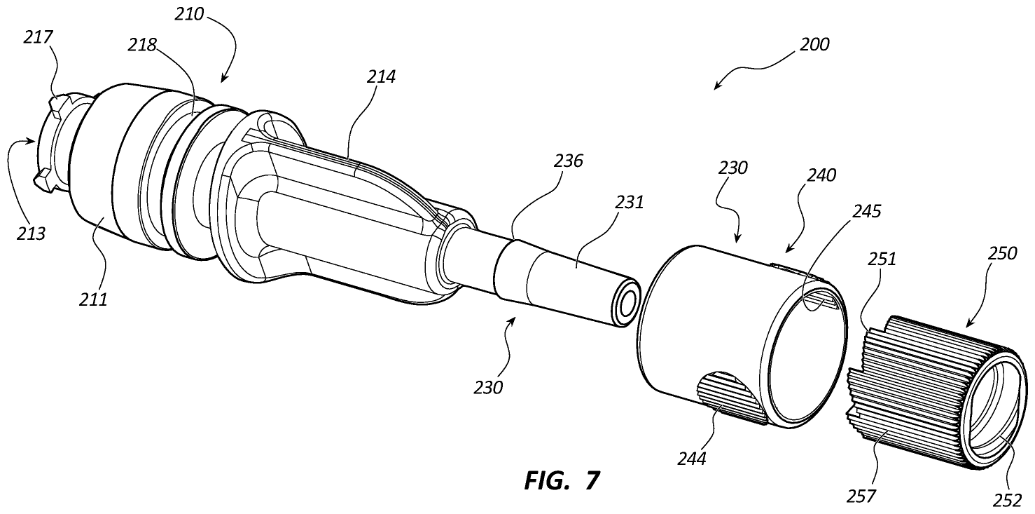

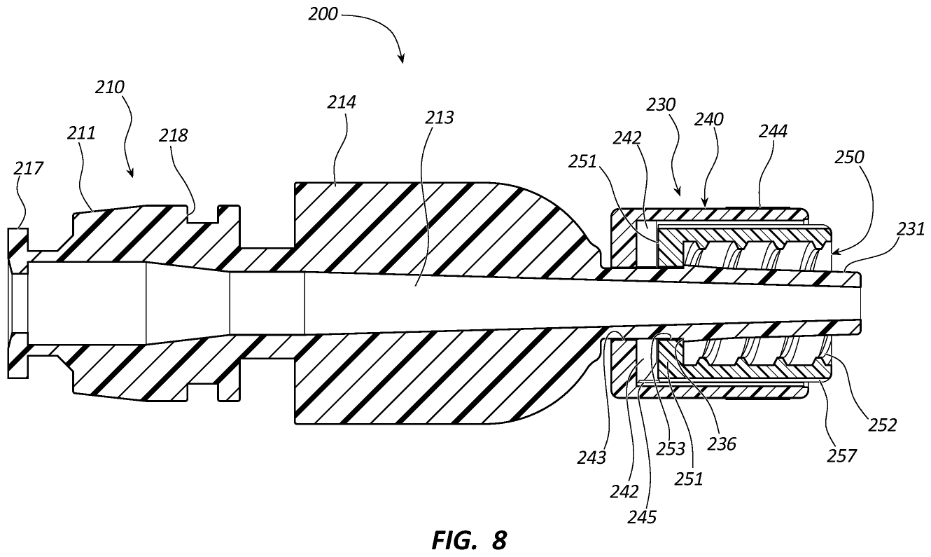

FIG. 7 depicts an exploded view of the luer adaptor 200, and FIG. 8 depicts a cross-sectional view of the luer adaptor 200. As depicted, the body 210 includes a proximal end 211 in fluid communication with a lumen 213. The lumen 213 may extend through the body 210 and the distal member 230 of the luer adaptor 200. In some embodiments, a portion of the lumen 213 adjacent the proximal end 211 may be configured as a female taper having a conical 4%-8% taper (e.g., 6% luer conical taper). This female taper can be configured to sealingly couple with a male taper of a medical delivery or drainage device. In certain embodiments, a valve member may be disposed within the lumen 213 (e.g., such as adjacent to the proximal end 211).

The proximal end 211 may be configured to engage with a medical device. In the illustrated embodiment, the proximal end 211 includes at least one laterally extending anti-rotation protrusion 217 configured to restrict rotation of a connected medical device relative to the luer adaptor 200. In another embodiment, the proximal end 211 may include threads or lugs configured to threadingly couple with a male luer lock fitting that includes an internally threaded collar. In further embodiments, the portion of the lumen 213 adjacent the proximal end 211 may be configured to couple with male protrusions of medical devices that are specifically configured for a particular medical therapy. For example, the lumen 213 may be configured to receive a male protrusion that is configured to open a valve disposed within the lumen 213. In another example, the lumen 213 may include a diameter that is configured to receive a male protrusion of a medical device that is configured for deliverance of a specific medicament or fluid. This configuration may reduce the incidence of accidental delivery of a wrong medicament or fluid to a patient.

The body 210 may further include a circumferential engagement groove 218. The engagement groove 218 may be configured to engage with a clip of a medical device to prevent inadvertent proximal displacement of the medical device from the luer adaptor 200. In the illustrated embodiment, the body 210 includes at least one laterally extending wing 214 configured to facilitate rotation of the body 210 and/or luer adaptor 200.

The distal member 230 of the illustrated embodiment may include one or more of a tapered protrusion 231, a spin nut shroud 240, and a spin nut 250. The tapered protrusion 231 may be configured as a male fitting having a 4%-8% conical taper (e.g., 6% luer conical taper) and may be configured to sealingly couple with a female fitting. The tapered protrusion 231 may include a shoulder 236 disposed proximally of a distal end. The shoulder 236 may be configured to restrict the spin nut shroud 240 and the spin nut 250 from distal displacement over the tapered protrusion 231. The tapered protrusion 231 can be fixedly coupled to the body 210. The tapered protrusion 231 may be coupled to the body 210 using any suitable technique. For example, the tapered protrusion 231 may be coupled to the body 210 by gluing, bonding, welding, friction fit, etc. In the illustrated embodiment, the body 210 and the tapered protrusion 231 are formed as an integral unit.

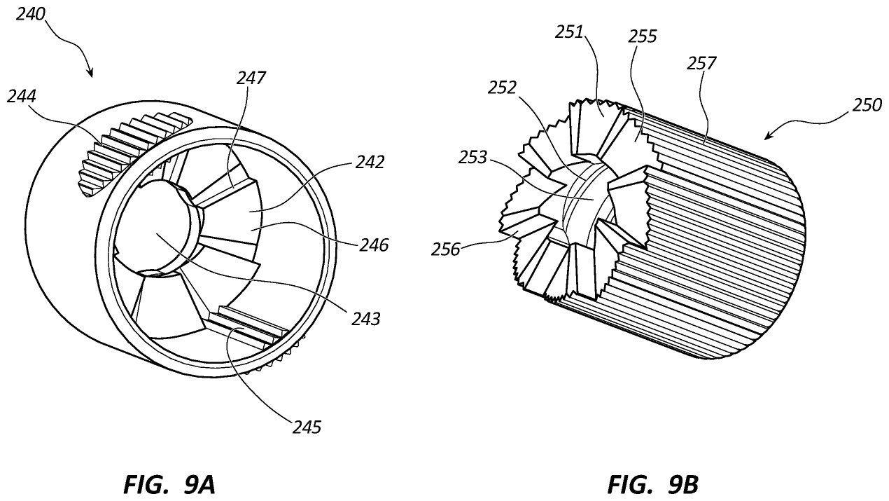

As illustrated in FIG. 9A, the spin nut shroud 240 is generally cylindrical in shape and can be disposed over the spin nut 250. The spin nut shroud 240 may comprise one or more of a pinch or compression member 244, shroud teeth 245, an optional shroud ramp 242, and a shroud passage 243. The pinch or compression member 244 may be configured to enhance pinchability of the spin nut shroud 240 when pinched (or compressed) by fingers of a user to facilitate engagement of the shroud teeth 245 with spin nut teeth 257. The pinch or compression member 244 can be disposed on an outer surface of the spin nut shroud 240 near a distal end. The pinch or compression member 244 may be of any suitable form. For example, as shown in the illustrated embodiment, the pinch or compression member 244 includes a plurality of longitudinally oriented ribs. In other embodiments, the pinch or compression member 244 can include a plurality of bumps or recesses, a textured surface, etc. The spin nut shroud 240 may include a plurality of pinch or compression members 244. For example, the spin nut shroud 240 may include one, two, three, four, or more pinch or compression members 244 disposed circumferentially about the outer surface of the spin nut shroud 240.

The shroud teeth 245 can be disposed on an internal surface of the spin nut shroud 240. The shroud teeth 245 may extend from a proximal end to the distal end of the spin nut shroud 240. In other embodiments, the shroud teeth 245 may be disposed adjacent the distal end of the spin nut shroud 240. The shroud teeth 245 may be at least partially positioned directly below the pinch or compression member 244. The spin nut shroud 240 can include a plurality of shroud teeth 245 configured to engage or mesh with spin nut teeth 257 when the spin nut shroud 240 is pinched (or compressed) at the pinch or compression member 244. The spin nut shroud 240 can include one, two, three, four, or more shroud teeth 245. The shroud teeth 245 may mesh with the spin nut teeth 257 to rotate the spin nut 250 when the spin nut shroud 240 is pinched (or compressed) and rotated.

An optional shroud ramp 242 may be disposed at a proximal end within the shroud 240 and circumferentially disposed around the shroud passage 243. The shroud ramp 242 may comprise a shroud ramp surface 246 and a shroud ramp shoulder 247.

The shroud ramp surface 246 may be a discontinuously curved helical surface (or a plurality of distinct helical surfaces extending around the spin nut shroud 240). The shroud ramp surface 246 and the shroud ramp shoulder 247 may be configured to engage with the spin nut surface (255 of FIG. 9B) and the spin nut shoulder (256 of FIG. 9B), respectively. Without limitation, the shroud ramp surface 246 may be angled distally at an angle from about 1 degree to about 45 degrees, from about 10 degrees to about 30 degrees, or from about 15 degrees to about 25 degrees. Other angles are also contemplated. Without limitation, the height of the shroud ramp shoulder 247 may range from about 0.01 inch to about 0.25 inch, from about 0.05 inch to about 0.20 inch, or from about 0.12 inch to about 0.15 inch. Other heights are also contemplated. The spin nut shroud 240 may comprise one, two, three, four, or more shroud ramps 242.

The shroud passage 243 may be disposed at the proximal end of the spin nut shroud 240. A diameter of the shroud passage 243 may be smaller than a diameter of the shoulder 236, such that the spin nut shroud 240 is restricted from distal displacement over the distal member 230. The diameter of the shroud passage 243 may be larger than a portion of the distal member 230 that is proximal to the shoulder 236, such that the spin nut shroud 240 is rotatable relative to the distal member 230.

As illustrated in FIG. 9B, the spin nut 250 is generally cylindrical in shape and can be disposed over the tapered protrusion 231. The spin nut 250 may comprise one or more of the spin nut teeth 257, an optional spin nut ramp 251, an internal thread 252, and a spin nut passage 253. The internal thread 252 may be configured to threadingly couple with external threads or protrusions of a female fitting. For example, the internal thread 252 may be a double-start, right handed threaded having between a 2-3 mm pitch (e.g., or about a 2.5 mm pitch).

The spin nut teeth 257 may be disposed circumferentially on an outer surface of the spin nut 250. The spin nut teeth 257 may extend from a proximal end to a distal end of the spin nut 250. In another embodiment, the spin nut teeth 257 may be disposed adjacent the distal end of the spin nut 250. The spin nut teeth 257 may be configured to mesh with the shroud teeth 245 to facilitate rotation of the spin nut 250 when the spin nut shroud 240 is rotated.

An optional spin nut ramp 251 may be disposed at a proximal end of the spin nut 250 and circumferentially disposed around the spin nut passage 253. The spin nut ramp 251 may comprise a spin nut ramp surface 255 and a spin nut ramp shoulder 256. The spin nut ramp surface 255 may be a discontinuously curved helical surface (or a plurality of distinct helical surfaces extending around the spin nut 250). The spin nut ramp surface 255 and the spin nut ramp shoulder 256 may be configured to engage with the shroud ramp surface (246 of FIG. 9A) and the shroud ramp shoulder (247 of FIG. 9A), respectively. Without limitation, the spin nut ramp surface 255 may be angled proximally at an angle from about 1 degree to about 45 degrees, from about 10 degrees to about 30 degrees, or from about 15 degrees to about 25 degrees. Other angles are also contemplated. The height of the spin nut ramp shoulder 256 may range from about 0.01 inch to about 0.25 inch, from about 0.05 inch to about 0.20 inch, or from about 0.12 inch to about 0.15 inch. Other heights are also contemplated. The spin nut 250 may comprise one, two, three, four, or more spin nut ramps 251.

The spin nut ramp 251 may be configured to engage with the shroud ramp 242 when the spin nut shroud 240 is rotated in a first direction to rotate the spin nut 250 in a first direction. Additionally, the spin nut ramp 251 may be configured to disengage from the shroud ramp 242 when the spin nut shroud 240 is rotated in a second direction to restrict the spin nut 250 from being rotated in the second direction. In other words, the spin nut ramp surface 255 and the spin nut ramp shoulder 256 are configured to engage with the shroud ramp surface 246 and the shroud ramp shoulder 247 when the spin nut shroud 240 is rotated in a first direction. Also, the spin nut ramp surface 255 and the spin nut shoulder 256 may be configured to disengage from the shroud ramp surface 246 and the shroud ramp shoulder 247 when the spin nut shroud 240 is rotated in the second direction. In other embodiments, the spin nut shroud 240 and the spin nut 250 may be free of ramps 242, 251. When free of ramps 242, 251 the spin nut shroud 240 may rotate the spin nut 250 in both the first and second directions when the shroud teeth 245 are engaged or meshed with the spin nut teeth 257.

The spin nut passage 253 may be disposed at the proximal end of the spin nut 250. A diameter of the spin nut passage 253 may be smaller than a diameter of the shoulder 236, such that the spin nut 250 is restricted from distal displacement over the distal member 230. The diameter of the spin nut passage 253 may be larger than a portion of the distal member 230 that is proximal to the shoulder 236, such that the spin nut 250 is rotatable relative to the distal member 230.

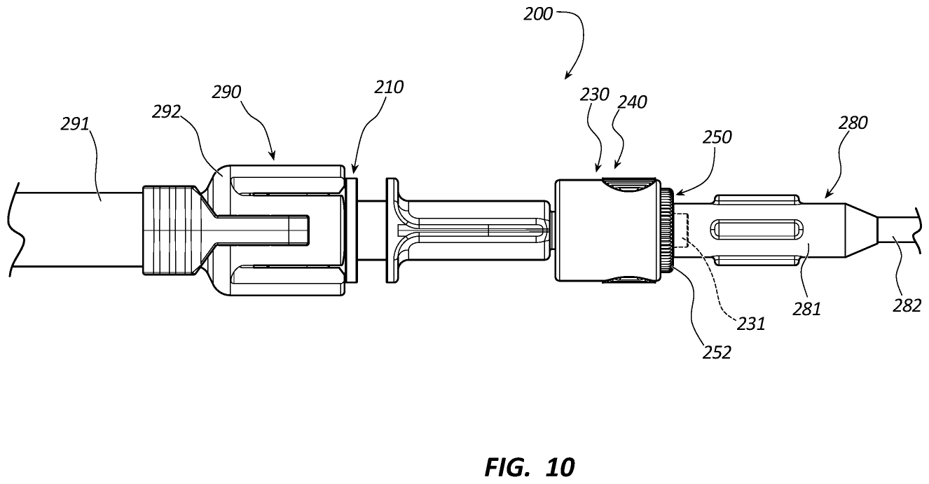

In use, the luer adaptor 200 may be utilized as a component of a fluid delivery or drainage system. In other embodiments, the luer adaptor 200 may be coupled to a pig tail drainage catheter, biliary drainage catheter, a nephrostomy tube, a central venous catheter, a peripheral intravenous catheter, a peripherally inserted central venous catheter, an epidural catheter, an intrathecal catheter, or another type of catheter or device. For example, as depicted in FIG. 10, a fluid drainage system 290 may be coupled to the body 210 such that the luer adaptor 200 is in fluid communication with the fluid drainage system 290. In other embodiments, a fluid delivery system may be coupled to the body 210. The fluid drainage system 290 may comprise a coupler 292, a drainage tube 291, and a drainage receptacle (not shown). Additionally, the drainage catheter 280, such as a pigtail catheter, may be coupled to the distal member 230. The drainage catheter 280 may comprise a catheter body 282 and a catheter hub 281 coupled to the catheter body 282. A distal portion of the catheter body 282 may be inserted into a fluid-filled cavity of a patient. The catheter hub 281 may be configured as a female luer lock fitting. The catheter hub 281 may be coupled to the distal member 230 such that the fluid drainage system 290 is in fluid communication with the fluid-filled cavity through the drainage catheter 280 and the luer adaptor 200.

The catheter hub 281 may be coupled to the distal member 230 when the tapered protrusion 231 is disposed within the catheter hub 281. The spin nut shroud 240 may be displaced distally to engage the optional shroud ramp 242 with the optional spin nut ramp 251. When the optional ramps 242, 251 are engaged, the spin nut shroud 240 may be rotated in the first direction by the user to rotate the spin nut 250 in the first direction. When the spin nut 250 is rotated in the first direction, the thread 252 can threadingly engage with the catheter hub 281, causing the catheter hub 281 to be drawn into the spin nut 250 and over the tapered protrusion 231 to fluidly seal the tapered protrusion 231 and the catheter hub 281.

In other embodiments, the catheter hub 281 can be coupled to the distal member 230 without utilization of the optional ramps 242, 251. In such embodiments, the spin nut shroud 240 can be pinched (or compressed) by the user such that the shroud teeth 245 engage or mesh with the spin nut teeth 257, and the spin nut shroud 240 can be rotated in the first direction causing the spin nut 250 to be rotated in the first direction.

In some embodiments, the luer adaptor 200 may be releasably coupled to the catheter hub 281. The spin nut shroud 240 may be pinched (or compressed) by the user causing the shroud teeth 245 to engage or mesh with the spin nut teeth 257. The spin nut shroud 240 can be rotated in a second direction to cause rotation of the spin nut 250 due to the engagement or meshing of the shroud teeth 245 with the spin nut teeth 257 even though the optional shroud ramp 242 is disengaged from the optional spin nut ramp 251. Rotation of the spin nut 250 in the second direction may result in unthreading of the spin nut 250 from the catheter hub 281, and uncoupling of the tapered protrusion 231 from the catheter hub 281.

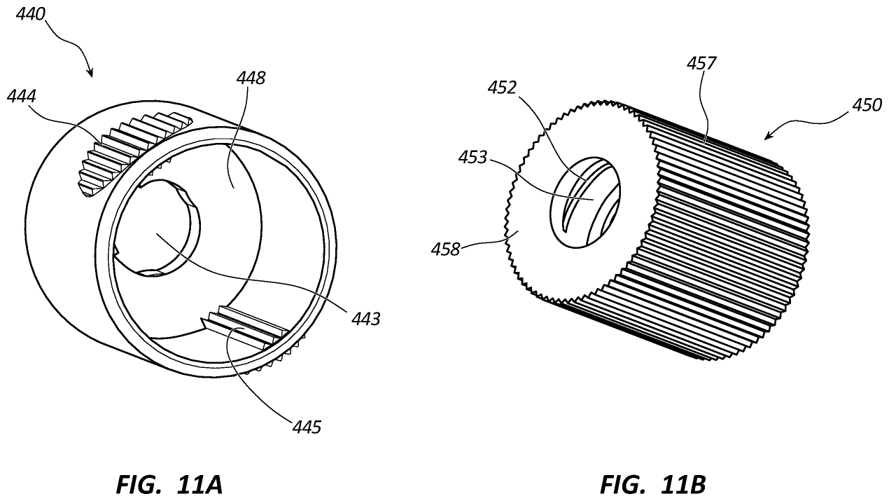

FIG. 11A illustrates an embodiment of a spin nut shroud 440. The spin nut shroud 440 is similar to the spin nut shroud 240 of the luer adaptor 200. The spin nut shroud 440 may include a shroud passage 443, a pinch or compression member 444, and shroud teeth 445. However, the spin nut shroud 440 may not include a shroud ramp. Rather, the spin nut shroud 440 may include a shroud proximal surface 448. The shroud proximal surface 448 may be flat and oriented perpendicular to a longitudinal axis of the spin nut shroud 440.

FIG. 11B illustrates and embodiment of a spin nut 450. The spin nut 450 may be similar to the spin nut 250 of the luer adaptor 200. The spin nut 450 may include an internal thread 452, a spin nut passage 453, and spin nut teeth 457. However, the spin nut 450 may not include a spin nut ramp. Rather, the spin nut 450 may include a spin nut proximal surface 458. The spin nut proximal surface 458 may be flat and oriented perpendicular to a longitudinal axis of the spin nut 450. When the spin nut shroud 440 and the spin nut 450 are coupled, a user may pinch (or compress) the pinch or compression member 444 to engage the shroud teeth 445 with the spin nut teeth 457 such that the spin nut 457 may be rotated in a first and/or second direction.

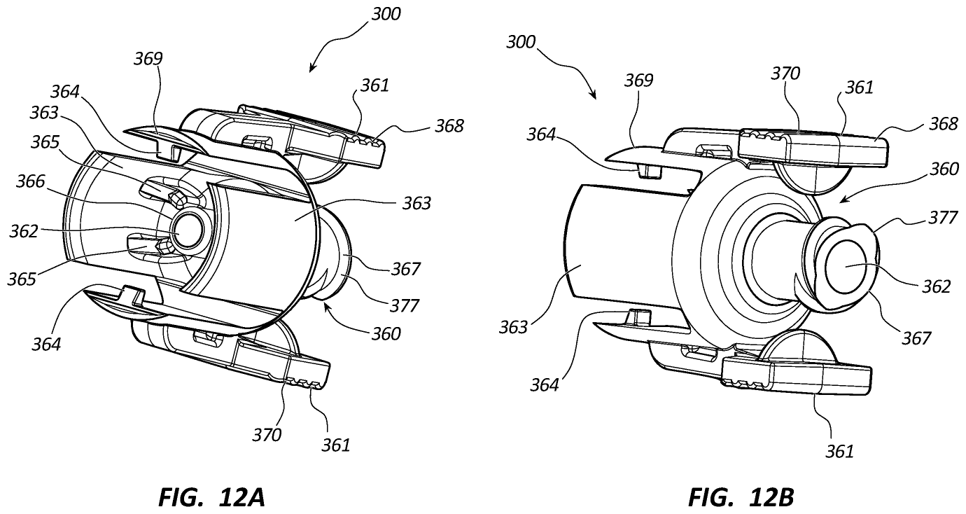

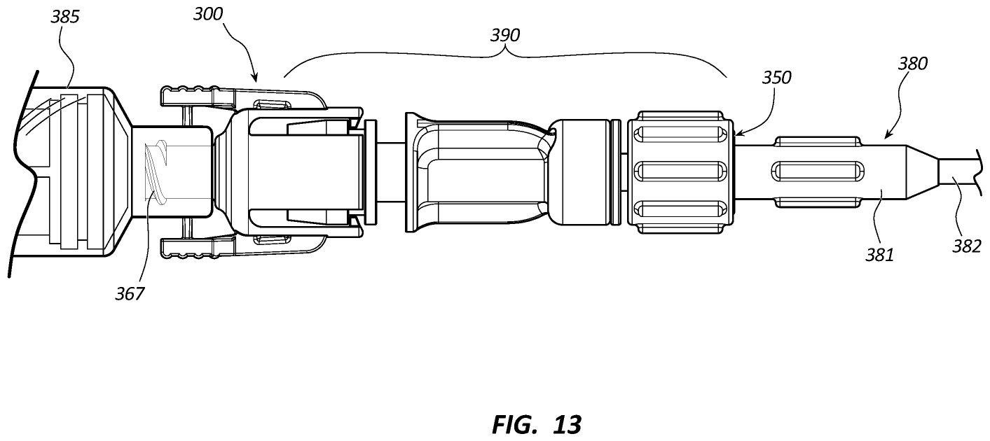

FIGS. 12A-12B depict an embodiment of a coupler 300. As illustrated in FIGS. 12A-12B, the coupler 300 includes one or more of a body 360, securement clips 361, a nozzle 366, and a female fitting 367. As depicted, the body 360 may have a cylindrical form with a lumen 362 extending through the body 360 from a proximal end to a distal end. The nozzle 366 may be disposed adjacent the distal end of the body 360. The nozzle 366 can have a cylindrical form, with the lumen 362 extending through the nozzle 366. The nozzle 366 may be configured to couple with a valved adaptor and to actuate the valve such that fluid may flow from the valved connector and through the coupler 300, or vice versa. For example, the nozzle 366 may actuate a valve of an Aspira drainage system when the coupler 300 is coupled to an Aspira connector. In other embodiments, the nozzle 366 may actuate a valve disposed within a luer adaptor, such as the luer adaptors previously described.

The female fitting 367 may be disposed adjacent the proximal end of the body 360. The lumen 362 may extend through the female fitting 367 such that the female fitting 367 is in fluid communication with the nozzle 366. The portion of the lumen 362 within the female fitting 367 may include a 4%-8% conical taper 9 (e.g., a 6% luer conical taper) and may be configured to sealingly couple with a male fitting. The female fitting 367 may comprise external threads or tabs 377. For example, the external threads 377 can include a double start, right-handed, and a 1-5 mm pitch (e.g., such as a 2.5 mm pitch). The external threads 377 may be configured to threadingly engage with internal threads of a male collar.

The clips 361 may comprise a proximal portion 368 and a distal portion 369. The clips 361 can be flexibly coupled to the body 360 between the proximal portion 368 and the distal portion 369. The proximal portion 368 may include grip features 370. The grip features 370 may allow for secure pinching (or compressing) of the clips 361 with fingers of a user. In the illustrated embodiment, the grip features 370 are shown as a plurality of transversely oriented ribs. In other embodiments, the grip features 370 may be any suitable feature, such as bumps, divots, a textured surface, a compliant surface, etc. The distal portion 369 may comprise inwardly directed engagement nubs 364. The engagement nubs 364 may be configured to engage with an engagement groove of an adaptor. For example, the engagement nubs 364 may engage with an engagement groove of the connector of the Aspira drainage system such that the coupler 300 may not be removed from the Aspira connector without pinching (or compressing) of the clips 361 by the user. In other embodiments, the nubs 364 may engage with an engagement groove of a luer adaptor, such as the luer adaptors previously described.

In certain embodiments, the coupler 300 may further include anti-rotation lugs 365 and guides 363 as shown in FIG. 12A. The anti-rotation lugs 365 may be directed distally alongside the nozzle 366. The anti-rotation lugs 365 may be configured to engage with anti-rotation protrusions of the Aspira connector. In other embodiments, the anti-rotation lugs 365 may engage with anti-rotation protrusions of a luer adaptor, such as the luer adaptors previously described. The guides 363 may extend distally from the body 360. The guides 363 may facilitate coupling of the coupler 300 to a connector, such as the Aspira connector. In other embodiments, the guides 363 may facilitate coupling of the coupler 300 to the luer adaptor, such as the luer adaptors previously described.

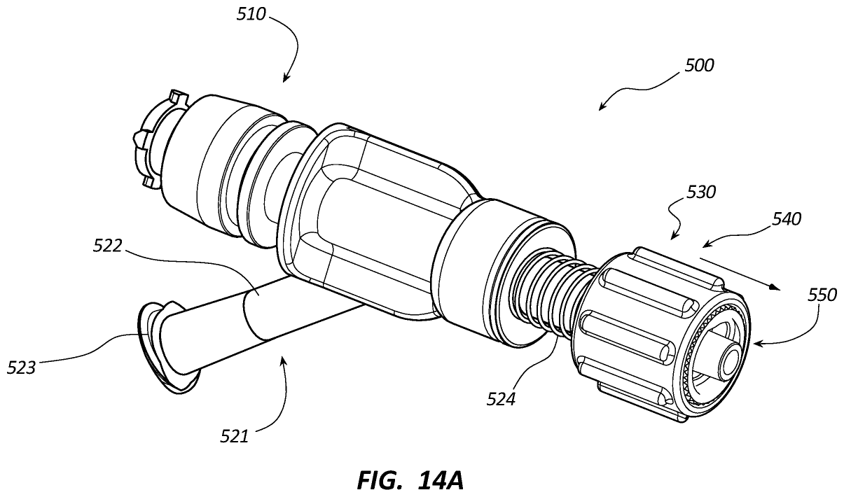

FIG. 13 depicts the coupler 300 in an exemplary use. As depicted, a medical device (e.g., syringe) 385 is coupled to the female fitting 367. The medical device 385 may be configured to deliver and/or withdraw fluid through the coupler 300. A luer adaptor 390, similar to the luer adaptors previously described, may be coupled to a distal end of the coupler 300. The coupler 300 may actuate a valve of the luer adaptor 390. The clips 361 may engage with an engagement groove of the luer adaptor 390 to retain the coupler 300 coupled to the luer adaptor 390. A catheter 380 may be coupled to a distal end of the luer adaptor 390. The catheter 380 may include a catheter body 382 and a catheter hub 381 coupled to the catheter body 382. The catheter hub 381 may be sealingly coupled to the luer adaptor 390. The catheter body 382 may be in fluid communication with the medical device 385 through the coupler 300 and the luer adaptor 390. A distal portion of the catheter body 382 may be inserted into a patient. For example, the distal portion may be inserted into a fluid-filled cavity such that the medical device 385 may withdraw fluid from the cavity. In other embodiments, the distal portion may be inserted into a blood vessel such that the medical device 385 may deliver a fluid and/or medicament into the blood vessel. Other devices may also be delivered to and/or from a patient via the coupler 300, including guide wires and the like.

In certain embodiments, the coupler 300 (or another portion of the luer adaptor 390) may comprise sensing elements. For example, the sensing elements may be configured to measure a fluid pressure, a fluid flow rate, and a chemistry of a fluid within the coupler 300. The sensing elements may provide the measurements to a monitoring device via a wireless connection. In other embodiments, the coupler 300 may include a flow regulator configured to regulate the fluid flow rate through the coupler 300, either for the controlled removal of fluid from a body cavity (e.g., slow fluid withdrawal from the pleural space in order to reduce the likelihood of re-expansion pulmonary edema) or the administration of medicines into the body cavity. For example, the flow regulator may be configured to instill tissue plasminogen activator (TPA) at a prescribed rate and or dose through the coupler 300 for the purpose or dissolving loculations within an abscess. The flow regulator may be controlled via a wireless connection to a remote control unit. In some embodiments, the coupler 300 may include a pressure sensor configured to detect a blockage in a catheter and to trigger an alarm to notify the patient or a clinician. In other embodiments, a pressure sensor may be employed to regulate the withdrawal of fluid from a body cavity such as the pleural space, based on the development of negative pressure within said body cavity, in order to lessen the risk for the development of re-expansion pulmonary edema.

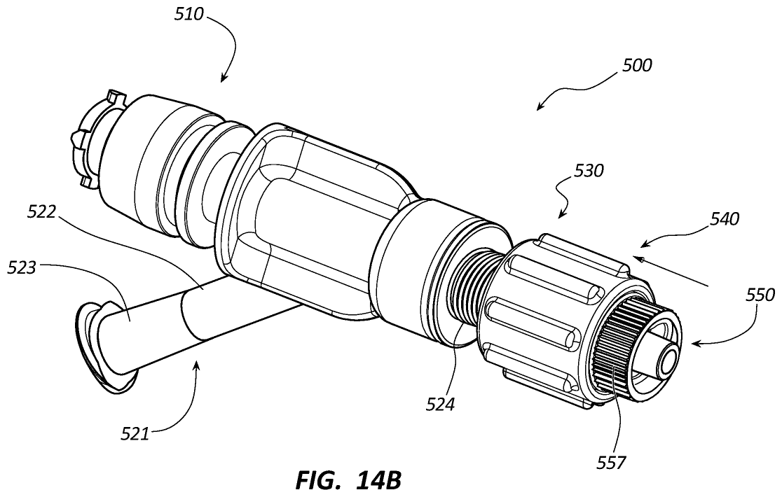

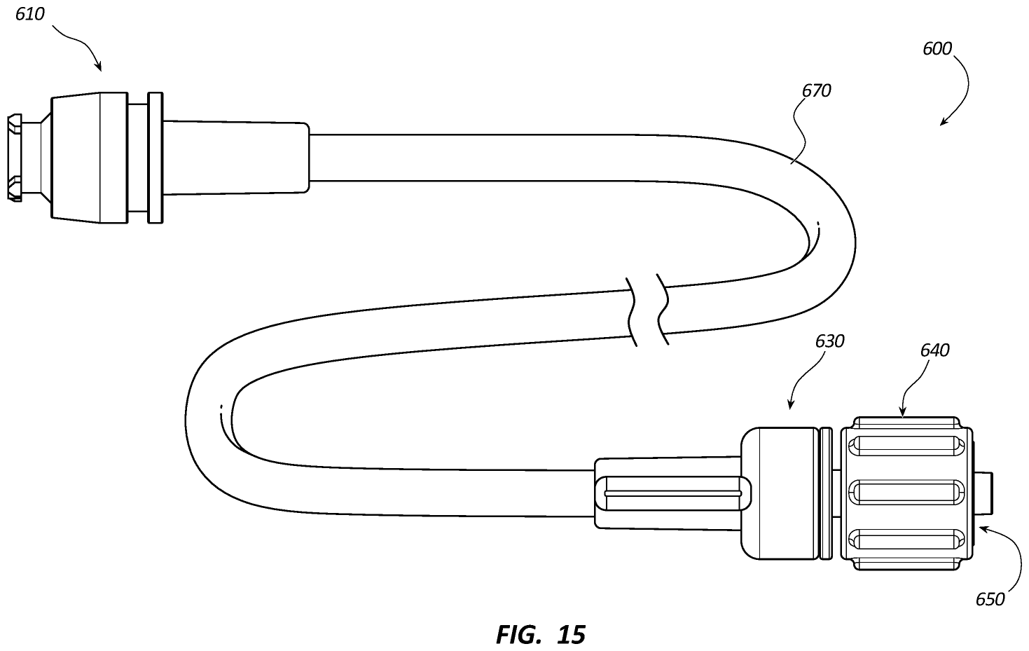

FIGS. 14A-14B depict another embodiment of a locking luer adaptor 500. The luer adaptor 500 is similar to the luer adaptors previously described having a body 510, a distal member 530, a spin nut shroud 540, and a spin nut 550. In the illustrated embodiment, the luer adaptor 500 can include any one of a resilient member 524 and a side port 521. The resilient member 524 may be any suitable type of compressible compliant element (e.g., compression spring). The resilient member 524 may be disposed around a portion of the distal member 530 and between the body 510 and the spin nut shroud 540 as shown in FIG. 14A. The spin nut shroud 540 may have a smooth internal surface and an exterior surface of the spin nut 550 may include gripping features 557, such as knurling, ribs, bumps, dimples, etc. The spin nut shroud 540 may be proximally displaceable relative to the spin nut 550 such that the resilient member 524 is compressed and the spin nut 550 is exposed, as illustrated in FIG. 14B. Exposure of the spin nut 550 may facilitate directly gripping of the spin nut 550 by the clinician to either rotate the spin nut 550 in a first direction and/or a second direction. Rotation of the spin nut 550 in the first direction may couple the luer adaptor 500 to a medical device while rotation of the spin nut 550 in the second direction may decouple the luer adaptor 500 from the medical device. The resilient member 524 may displace the spin nut shroud 540 distally when the spin nut 550 is released by the clinician to shield the spin nut 550 from inadvertent rotation.

The side port 521 may be fixedly coupled to the body 510. The side port 521 may extend laterally from the body 510 at an angle ranging from about 15 degrees to about 90 degrees (e.g., about 45 degrees). The side port 521 may be formed of a rigid, semi-rigid, or flexible polymer. In certain embodiments, the side port 521 is formed from the same material as the body 510. The side port 521 may include an arm portion 522 and a connector portion 523. The arm portion 522 may include a lumen that is in fluid communication with a lumen of the body 510. The connector portion 523 may be disposed adjacent a free end of the arm 522 and be configured to couple with a fluid transfer device (e.g., male luer fitting).

FIG. 15 illustrates another embodiment of a locking luer adaptor 600. As depicted a flexible extension tube 670 may be sealingly coupled to and disposed between a body 610 and a distal member 630 (which can include a spin nut 650 and spin nut shroud 640). A lumen of the extension tube 670 may be fluid communication with a lumen of the body 610 and the distal member 630. The extension tube 670 may allow for easier access to the body 610 by a user when connecting a medical device to the body 610.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. Further, all ranges include both endpoints.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. The scope of the invention is therefore defined by the following claims and their equivalents.

1. A fluid connector, comprising:
a proximal body;
a tapered protrusion coupled to and extending distally from the proximal body, wherein a lumen extends through the proximal body and the tapered protrusion;
a spin nut rotatably disposed over at least a portion of the tapered protrusion; and
a spin nut shroud disposed over the spin nut;
wherein the spin nut shroud is configured to engage with the spin nut when rotated in a first direction and to disengage from the spin nut when rotated in a second direction.

2. The fluid connector of claim 1, wherein the proximal body comprises a valve disposed in the lumen.

3. The fluid connector of claim 1, wherein the tapered protrusion comprises a shoulder configured to retain the spin nut disposed partially over the tapered protrusion.

4. The fluid connector of claim 1, wherein the spin nut and the spin nut shroud are rotatable relative to the tapered protrusion.

5. The fluid connector of claim 4, wherein the spin nut comprises nut teeth, and the spin nut shroud comprises shroud teeth, and wherein the nut teeth are configured to engage with the shroud teeth to rotate the spin nut relative to the tapered protrusion when the spin nut shroud is rotated.

6. The fluid connector of claim 1,
wherein the spin nut comprises a nut ramp, and the spin nut shroud comprises a shroud ramp; and
wherein the shroud ramp is configured to engage the nut ramp when the spin nut shroud is rotated in the first direction and to disengage the nut ramp when the spin nut shroud is rotated in the second direction.

7. The fluid connector of claim 1, wherein the proximal body is rotatable about a longitudinal axis relative to the tapered protrusion.

8. The fluid connector of claim 1, further comprising:
a locking collar; and
an O-ring;
wherein the locking collar couples the tapered protrusion to the proximal body at a rotatable joint; and
wherein the O-ring seals the rotatable joint.

9. The fluid connector of claim 1, further comprising a flexible tube disposed between the proximal body and the tapered protrusion, wherein the proximal body is coupled to one end of the flexible tube, and the tapered protrusion is coupled to an opposite end of the flexible tube, and wherein the proximal body is in fluid communication with the tapered protrusion.

10. The fluid connector of claim 1, wherein the spin nut shroud is configured to be proximally displaceable relative to the spin nut, wherein the spin nut is exposed.

11. The fluid connector of claim 10, further comprising a compliant member disposed between the spin nut shroud and the proximal body and configured to displace the spin nut shroud distally, wherein the spin nut is surrounded by the spin nut shroud.

12. A fluid transfer system, comprising:
a fluid connector, comprising:
a proximal body configured to be connectable to a first fluid fitting;
a tapered protrusion coupled to the proximal body, wherein the tapered protrusion is connectable to a second fluid fitting;
a rotatable spin nut disposed over at least a portion of the tapered protrusion; and
a spin nut shroud disposed over the spin nut;
wherein the spin nut shroud is configured to engage with the spin nut when rotated in a first direction and to disengage from the spin nut when rotated in a second direction; and
a coupler configured to couple with the proximal body.

13. The fluid transfer system of claim 12, wherein the spin nut comprises nut teeth, and the spin nut shroud comprises shroud teeth, and wherein the nut teeth are configured to engage with the shroud teeth to rotate the spin nut relative to the tapered protrusion when the spin nut shroud is rotated.

14. The fluid transfer system of claim 12,
wherein the spin nut comprises a nut ramp, and the spin nut shroud comprises a shroud ramp; and
wherein the nut ramp is configured to engage the shroud ramp when the spin nut shroud is rotated in the first direction and to disengage the shroud ramp when the spin nut shroud is rotated in the second direction.

15. The fluid transfer system of any one of claim 12, wherein the proximal body is rotatable about a longitudinal axis relative to the tapered protrusion.

16. The fluid transfer system of claim 12, wherein the coupler comprises:
a flexible clip configured to couple with the proximal body;
a valve actuator configured to open a valve disposed within a lumen of the proximal body; and
a proximal fitting configured to be in fluid communication with a medical device.

17. A method of transferring fluid, comprising:
obtaining a fluid connector, wherein the fluid connector comprises:
a proximal body;
a tapered protrusion extending from the proximal body;
a spin nut disposed over at least a portion of the tapered protrusion; and
a spin nut shroud disposed over the spin nut; and
connecting a fluid transfer catheter to the tapered protrusion.

18. The method of claim 17, wherein connecting the fluid transfer catheter to the tapered protrusion comprises:
rotating the spin nut shroud in a first direction relative to the tapered protrusion, wherein the spin nut shroud engages with the spin nut to rotate the spin nut in the first direction relative to the tapered protrusion and the fluid transfer catheter.

19. The method of claim 17, wherein the proximal body is rotatable relative to the tapered protrusion about a longitudinal axis, and the spin nut is un-rotatable in a second direction.

20. The method of claim 17, further comprising:
connecting a coupler to the proximal body; and
connecting a medical device to the coupler.
